(19) United States
(12) Patent Application Publication
(10) Pub. No.: US 2025/0312563 A1
(43) Pub. Date: Oct. 9, 2025

(54) SLEEP MONITORING AND STIMULATION PROVIDING SYSTEM USING MOBILE DEVICE, AND METHOD THEREOF

(71) Applicant: BRLAB INC, Seoul (KR)

(72) Inventors: Hyunbin KWON, Seoul (KR); Jongmin LEE, Seoul (KR); Kwangsuk PARK, Seoul (KR); Byunghun CHOI, Seoul (KR); Jonghyeok JEONG, Seoul (KR)

(21) Appl. No.: 19/245,133

(22) Filed: Jun. 20, 2025

Related U.S. Application Data
(63) Continuation of application No. PCT/KR2023/021432, filed on Dec. 22, 2023.

(30) Foreign Application Priority Data
Dec. 22, 2022 (KR) 10-2022-0182003
Dec. 22, 2022 (KR) 10-2022-0182004
Apr. 10, 2023 (KR) 10-2023-0047108
Dec. 22, 2023 (KR) 10-2023-0189306

(51) Int. Cl. A61M 21/02; A61B 5/00; A61M 21/00; G16H 20/70; G16H 40/67

(52) U.S. Cl. CPC A61M 21/02; G16H 20/70; G16H 40/67; A61B 5/4806; A61M 2021/0022; A61M 2205/3303

(57) ABSTRACT

The present disclosure relates to a sleep monitoring and stimulation providing system using a mobile device, and a method thereof.

### CROSS-REFERENCE TO RELATED APPLICATIONS

This application is a Continuation of Application No. PCT/KR2023/021432, filed on Dec. 22, 2023, which in turn claims the benefit of Korean Patent Applications No. 10-2022-0182003, filed on Dec. 22, 2022, No. 10-2022-0182004, filed on Dec. 22, 2022, No. 10-2023-0047108, filed on Apr. 10, 2023, and No. 10-2023-0189306, filed on Dec. 22, 2023. The entire disclosures of all these applications are hereby incorporated by reference.

### TECHNICAL FIELD

The present disclosure relates to a sleep monitoring and stimulation providing system using a mobile device, and a method thereof.

### BACKGROUND ART

Recently, as people's living standards and quality have improved, the demand for ‘good sleep’ has increased, and in particular, the industry that provides various sleep-inducing devices or services using the latest scientific technologies is growing significantly. Accordingly, many devices and services are being commercialized to improve sleep quality, such as sleep care services that provide guidance on sleep environment, habits, and posture through consulting with experts, and services that monitor a sleep state by detecting a user's breathing sounds, or the like when wearing a wearable device. Specifically, various devices and services are being developed that receive information such as the user's physical information, state information, and sleep habits, or collect information such as the user's state information or sleep habits through separate equipment, and function to induce ‘good sleep’ based on such information on the user, thereby inducing a sleep state based on user-specific state information.

In order to accurately identify the user's sleep state, it is essential to have equipment that measures the user's state information or biometric information, and to this end, the user has no choice but to wear separate equipment, and wearing such separate equipment inevitably causes inconvenience while the user sleeps. In other words, in order to induce the user's ‘good sleep’, it is important to more accurately identify the user's sleep state, but, in the case of the prior art, there is a limitation that the user's ‘good sleep’ is inevitably disturbed due to a separate device worn by the user to identify the user's sleep state.

The present disclosure has been proposed to overcome the limitations of the prior art, and aims to provide a sleep monitoring and stimulation providing system using a mobile device, which can more accurately monitor a user's sleep state and provide improvement measures without disturbing the user's sleep, and a method thereof.

### DISCLOSURE OF INVENTION

### Technical Problem

An aspect of the present disclosure is to provide a sleep monitoring and stimulation providing system using a mobile device, which can accurately determine a user's sleep state by collecting the user's biometric information through a mobile device provided in a structure such as a mattress on which the user sleeps, and a method thereof.

In addition, another aspect of the present disclosure is to utilize a vibration means provided in a mobile device to cause the mobile device provided in a mattress to generate vibration stimulation while the user is sleeping, thereby influencing the user's sleep state.

Moreover, still another aspect of the present disclosure is to provide a sleep monitoring and stimulation providing system using a mobile device, which can collect a user's biometric information using a mobile device located on the user or a mattress on which the user sleeps, and monitor the user's sleep state based on the collected user's biometric information or provide stimulation that can improve the user's sleep state, and a method thereof.

Meanwhile, technical problems of the present disclosure are not limited to the above-mentioned problems, and other technical problems which are not mentioned herein will be clearly understood by those skilled in the art from the description below.

### Technical Solution

In order to solve the foregoing problems, a sleep analysis segment detection system using a mobile device according to the present disclosure may include at least one layer having a plurality of components; a detection unit for detecting a biosignal of a user lying on the layer unit; a mounting unit located inside or outside the layer unit to mount a mobile device; a memory unit in which the user's biosignal collected by the detection unit is stored; and a calculation unit that analyzes the user's sleep state based on the user's biometric information stored in the memory unit, wherein the calculation unit, the detection unit, and the memory unit are operated while the mobile device mounted on the mounting unit is in conjunction with the calculation unit.

In addition, in the system, the mobile device may be provided in a form that can be mounted and detached on a surface or inside of the mounting unit.

Meanwhile, a sleep monitoring and stimulation providing system using a mobile device according to another embodiment of the present disclosure may include at least one layer unit; and a mounting unit located inside the layer unit and configured to mount a mobile device, wherein a biosignal of a user lying on the layer unit is detected by the mobile device mounted on the mounting unit, or vibration stimulation is generated in a direction of an upper surface of the layer unit.

In addition, in the system, the mounting unit may be provided at a position in contact with an uppermost surface of the layer unit.

Meanwhile, a sleep monitoring and stimulation providing method using a mobile device according to still another embodiment of the present disclosure, which is performed by a sleep monitoring and stimulation providing system using a mobile device including a layer unit, a detection unit for detecting a user's biosignal, a mobile device mounted on a mounting unit, a memory unit, and a calculation unit, the method may include collecting biometric information of a user lying on the layer unit by the detection unit; storing the user's biometric information in the memory unit; analyzing the user's sleep state in the calculation unit based on the user's biometric information stored in the memory unit; and vibrating the mobile device to generate vibration stimulation according to the user's sleep state.

In addition, a sleep monitoring and stimulation providing method using a mobile device according to yet still another embodiment of the present disclosure, which is performed by a sleep monitoring and stimulation providing system using a mobile device including a layer unit and a mobile device mounted on a mounting unit, the method may include collecting biometric information of a user lying on the layer unit by the mobile device; storing the user's biometric information in the mobile device; analyzing the user's sleep state based on the user's biometric information by the mobile device; and vibrating the mobile device according to the user's sleep state to generate vibration stimulation.

### Advantageous Effects

According to the present disclosure, a user's biometric information may be collected through a mobile device provided in a structure such as a mattress on which the user sleeps to determine the user's sleep state, thereby more accurately monitoring the user's sleep state without disturbing the user's sleep.

In addition, the present disclosure may utilize a vibration means provided in a mobile device even if the mattress is not originally manufactured for sleep improvement, thereby having an effect of providing vibration stimulation to a user during sleep.

Moreover, the present disclosure may repeatedly collect a user's biometric information periodically/aperiodically using a mobile device located on the user or a mattress on which the user sleeps, and provide a stimulation signal that can induce a ‘good sleep’ state according to the user's sleep state determined based on the collected biometric information, thereby providing a continuous sleep state monitoring and sleep state improvement method even while the user is sleeping.

Furthermore, the present disclosure may allow accurate sleep state analysis and optimal stimulation signal generation for each user since the user's biometric information collected from a mobile device is mapped and stored for each user, thereby allowing user-specific sleep monitoring and improvement using the mobile device, just by lying and sleeping on a mattress shared by a plurality of users, individually or together.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1 is a diagram for explaining a sleep monitoring

and stimulation providing system using a mobile device according to a first embodiment of the present disclosure.

FIG. 2A and FIG. 2B show a configuration provided with a mounting unit and a mobile device.

FIG. 3 shows a configuration further provided with a vibration assisting member.

FIG. 4 is a diagram for explaining a mobile device in more detail.

FIG. 5 is a diagram for explaining the memory unit in more detail.

FIG. 6 is a diagram for explaining a sleep monitoring and stimulation providing system using a mobile device according to a second embodiment of the present disclosure.

FIG. 7 shows a configuration provided with a mounting unit and a mobile device in the second embodiment.

FIG. 8 is a diagram for explaining a sleep monitoring and stimulation providing system using a mobile device according to a third embodiment of the present disclosure.

FIG. 9 is a diagram for explaining a sleep monitoring and stimulation providing method using a mobile device according to an embodiment of the present disclosure.

FIG. 10 is a diagram for explaining a sleep monitoring and stimulation providing method using a mobile device according to another embodiment of the present disclosure.

FIG. 11 is a diagram for explaining a sleep monitoring and stimulation providing method using a mobile device according to still another embodiment of the present disclosure.

FIG. 12 is a diagram for more specifically explaining a sleep monitoring and stimulation provision method using a mobile device according to still another embodiment of the present disclosure.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type”, and the expression simply denotes that the elements are present, but should not be construed as excluding additional elements. Moreover, in case where it is mentioned that one element is “connected” or “coupled” to the other element, it should be understood that one element may be directly connected to the other element, but another element may be present therebetween.

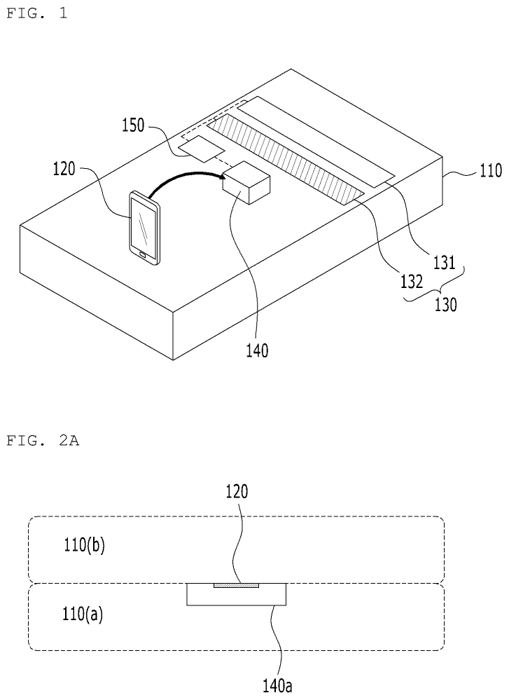

FIG. 1 is a diagram for explaining a sleep monitoring and stimulation providing system using a mobile device according to a first embodiment of the present disclosure.

Referring to FIG. 1, a sleep monitoring and stimulation providing system 100 using a mobile device according to the present disclosure may include at least one layer unit 110 having a plurality of components, and a mobile device 120 located inside or outside the layer unit 110 to collect and store user biometric information, and further analyze the user's sleep state based on the stored user biometric information, and further, a detection unit 130 including a vibration detection unit 131 for detecting a vibration signal and/or a pressure detection unit 132 for detecting a pressure signal may be provided on the layer unit 110, and furthermore, a calculation unit 150 in conjunction with the mobile device 120 to generate a signal for controlling the entire system 100 may be included within the system 100.

The system 100 according to the first embodiment may be implemented such that, simply put, the system 100 can be operated to perform sleep monitoring and/or stimulation providing functions only when the user mounts a smartphone in the mounting unit 140 while both the detection unit 130 and the calculation unit 150 are mounted on a mattress. Hereinafter, respective detailed components will be described.

The layer unit 110 may be understood as a member having a receiving space in which the components to be described later can be arranged, and if a receiving space is provided, there is no limitation on the material or shape of the layer unit 110. The layer unit 110 may be implemented in a form in which a plurality of things are accumulated, and ultimately may be a space where a user sleeps, for example, a mattress. Additionally, according to an embodiment, the layer unit 110 may be a mat that may be placed on a mattress, and may furthermore be a member made of wood or metal rather than cotton with a fiber material. In this manner, if the layer unit 110 has a predetermined receiving space, there is no limitation on the material or shape. However, in order to help understand the disclosure, in this detailed description, the explanation will be continued assuming that the layer unit 110 is a mattress.

The mobile device 120 may be located inside or outside the layer unit 110, and may control the system 100 in conjunction with the system 100 or receive signals acquired by the system 100, more precisely, the detection unit 130. In addition, if necessary, the mobile device 120 may provide vibration stimulation to a user lying on a mattress by vibrating the layer unit 110 using its own vibration means. In this case, the mobile device 120 may be provided in a form that can be mounted to and detached from a surface or inside of the layer unit. Preferably, the mobile device 120 may be located in an arbitrary mounting unit 140 disposed inside the layer unit 110, or may be located in an arbitrary mounting unit 140 provided outside the layer unit 110 so as to function to detect and collect user biometric information and function as a stimulation source that provides a stimulation signal to the user. In addition, since the mobile device 120 has a relatively small size and shape, it may be provided inside a mounting unit 140 manufactured with a special structure to protect/mount the mobile device 120 prior to being located in the storage box, if necessary. Here, the case may be preferably manufactured with a material, thickness, and shape that allow the user's biosignal to be effectively detected and collected through the detection unit of the mobile device 120. For reference, the mounting unit 140 shown in FIG. 1 is shown as being provided inside the layer unit 110.

As an example of the design of the mounting unit 140 for positioning a mobile device, the mounting unit 140 may be designed as a drawer type that can be opened and closed on a side surface of the mattress so as to allow the mobile device, that is, a smartphone, to be located at an arbitrary position within an internal space of the mattress, and may be designed so as to allow, when the drawer is closed, the mobile device 120 to be placed at a predetermined space within the mattress, more precisely, in the space where the layer unit 110 is present. Alternatively, the position and structure may be designed so as to be covered by an upper outer fabric of the mattress, and the user may open a cover of the mounting unit 140 provided inside the mattress as if opening a lid and mount the mobile device 120 therein. As another example, the mounting unit 140 may be designed and provided to be necessarily present outside the mattress, for example, the mounting unit 140 capable of mounting the mobile device 120 may be disposed to be dockable in a bed frame on which the mattress is placed so as to allow the user to easily connect his or her mobile device (smartphone) to the sleep monitoring and stimulation system. For reference, in the above description, the meaning of the mobile device 120 being mounted in the mounting unit 140 may refer to not only that the mobile device 120 is simply placed in the mounting unit 140, but also that the mobile device 120 is connected to the sleep monitoring and stimulation providing system in a wired or wireless manner and is in a state where it can send and receive control commands or information. Here, for reference, the mobile device 120 refers to a computer device having a shape, size, and weight that can be easily carried by a user while being provided or mounted thereon, and generally may include a display screen with a touch input, a small keyboard, a microphone, a speaker, and various types of sensors. For example, the mobile device 120 may include a terminal such as a mobile phone, a personal digital assistant (PDA), a mobile computer, a digital camera, a digital camcorder, or a wireless pager. That is, the sleep monitoring and stimulation providing system according to an embodiment of the present disclosure may use any small terminal that can detect and collect a user's biometric information, and the type of the mobile device 120 or the position, shape, method, and the like of the mobile device 120 may not be limited to the embodiment of the present disclosure.

Referring again to FIG. 1, the system 100 according to a first embodiment is provided with the detection unit 130, wherein the detection unit 130, which is provided on the layer unit 110, is configured to detect and acquire a biosignal from a user. The detection unit 130 may include one of various types of sensors capable of detecting a user's biosignal, for example, a pressure sensor, a vibration sensor, a piezoelectric sensor, an acceleration sensor, an acoustic sensor, a polyvinylidene film (PVDF) sensor, an electromechanical film (EMFi) sensor, an infrared sensor, a motion sensor, and a facial recognition sensor, and may be configured with a combination of at least one or more of the sensors.

Specifically, the detection unit 130 may include the pressure detection unit 132 that detects the user's operation of lying down or getting up from the layer unit 110 or the user's weight or position information through a pressure sensor, or the vibration detection unit 131 that detects the user's vibration signal through a vibration sensor, and state information such as a heart rate, a breathing state, and a movement state may be acquired through those detection units. In this way, the detection unit 130 of the present disclosure may be a component for detecting various biosignals of a user located on the layer unit 110, and adjusting the type, number, layout, and the like of sensors used to detect more precise and specific user state information cannot be limited to an embodiment of the present disclosure.

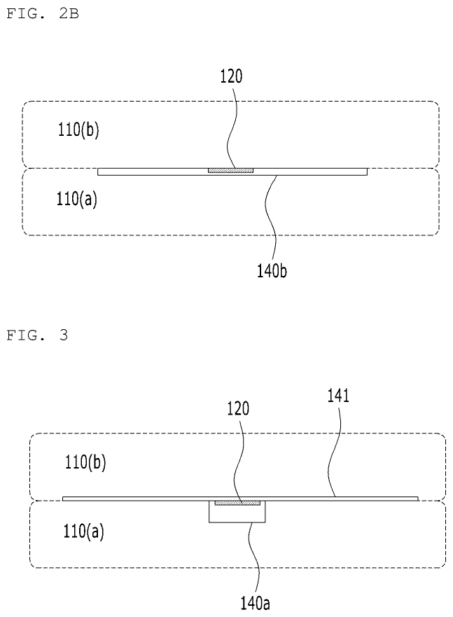

In the foregoing description, it has been mentioned that the mobile device 120 can be mounted in the mounting unit 140, and an exemplary structure is shown in FIGS. 2A and 2B. FIGS. 2A and 2B show cross-sectional views of a mattress, wherein there is shown a configuration in which the mounting unit 140 is provided so as to allow the mobile device 120 to be located inside the mattress, that is, between a first layer 110(a) and a second layer 110(b). The mobile device 120 may be mounted within the mounting unit 140, and preferably, the mounting unit 140 may be customized to the mobile device 120 so as to allow the mobile device 120 to be stably mounted in a fixed position as much as possible. In addition, the mounting unit 140 may not necessarily have to be a box-shaped structure with all six sides closed, and may have an open upper surface. When the box-shaped structure is closed on all six sides, the mobile device 120 may be located in close contact with an upper surface of the mounting unit 140, thereby allowing vibration stimulation to be transmitted toward an upper surface of the mattress through the mounting unit 140. When the upper surface of the mounting unit 140 is not present (open), the mobile device 120 may be located in close contact with a facing surface of the layer 110(b), thereby allowing vibration stimulation to be transmitted toward the upper surface of the mattress through the layer 110(b).

Meanwhile, the mounting unit 140 may not only be used for the purpose of accommodating the mobile device 120 thereinside, but may also be utilized to transmit vibration toward the upper surface of the mattress when the mobile device 120 vibrates. The mobile device 120 may have its own vibration means, and the mounting unit 140 for providing vibration stimulation to the user may be made of wood, metal, or synthetic resin so as to allow the vibration stimulation generated by the mobile device 120 to be further transmitted to the user.

In addition, the mounting unit may determine the properties of vibration by varying the length of each side of the hexahedron, as in 140a and 140b of FIGS. 2A and 2B. For example, the mounting unit 140a as in 140a may be designed to transmit vibration stimulation with a higher frequency (shorter cycle) to a user over a relatively narrow area, and 140b may be designed, conversely, to transmit vibration stimulation with a lower frequency (longer cycle) to a user over a relatively wider area.

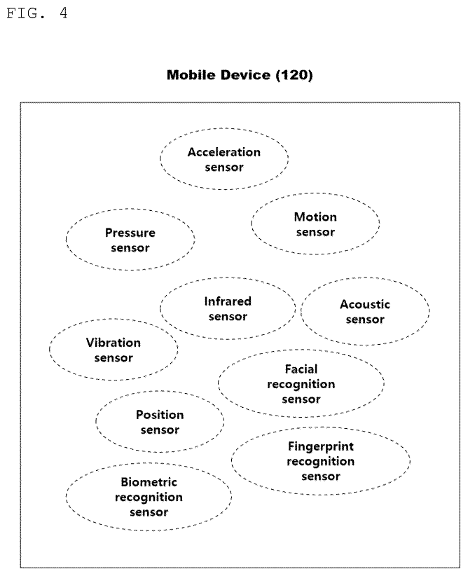

On the other hand, referring to FIG. 3, a vibration assisting member 141 may be further provided on an upper surface of the mounting unit 140 and the mobile device 120. A vibration signal generated by the mobile device 120 may not be fully transmitted to the user due to its small size, but the vibration assisting member 141 may be further placed on the upper surface of the mounting unit 140 and the mobile device 120, thereby reducing a decrease in vibration energy during the transmission process. A position provided with the vibration assisting member 141 may preferably be between layers, and in particular, the vibration assisting member 141 may be located on a surface that comes into contact with a point where a vibration signal is generated by the mobile device 120, thereby securing a relatively larger space in which the vibration assisting member 141 can be inserted, while at the same time achieving the effect of increasing the vibration transmission effect.

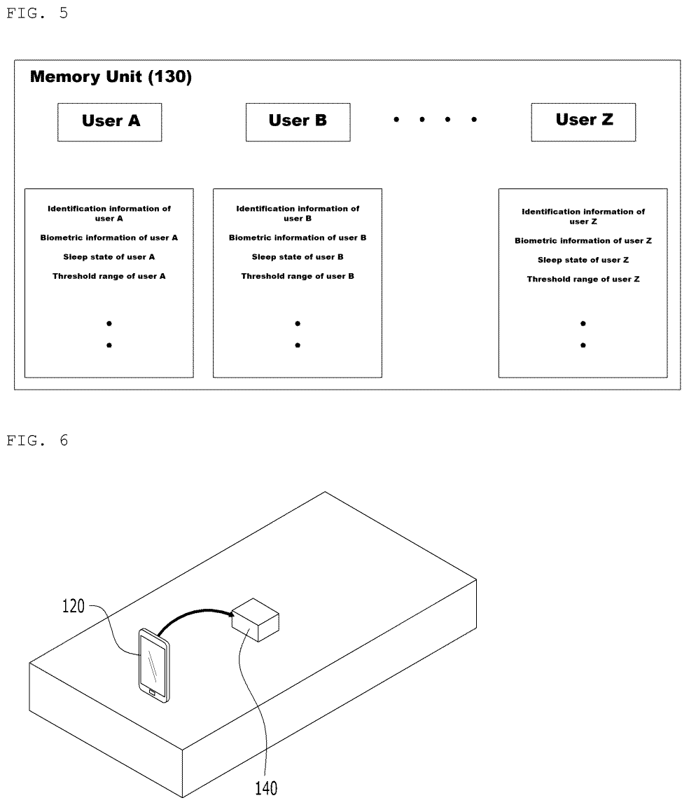

FIG. 4 is a diagram for more specifically explaining the mobile device 120 that can be used in a sleep monitoring and stimulation providing system according to an embodiment of the present disclosure, and as shown in FIG. 4, the mobile device 120 may include at least one or more sensors among various types of built-in sensors, for example, a pressure sensor, a vibration sensor, a position sensor, an acoustic sensor, an infrared sensor, a motion sensor, a facial recognition sensor, a biometric recognition sensor, and a fingerprint recognition sensor.

As will be described later, the mobile device 120 may detect the user's weight or location information through a pressure sensor, or detect the user's vibration signal through a vibration sensor to acquire state information such as a heart rate, a breathing state, and a movement state, and those sensor functions may be utilized to monitor the user's sleep state by being mounted on a mattress (layer) that is not originally provided with detailed components for sleep monitoring. In addition, the mobile device 120 may recognize the user's voice through an acoustic sensor and may detect the user's gesture through a motion sensor to receive movement information, and those sensor functions may also be utilized to monitor the user's sleep state.

FIG. 5 is a diagram for explaining in more detail the memory unit briefly mentioned above.

Referring to FIG. 5, the memory unit, which is a component in which collected user biometric information (pressure signal, vibration signal, etc.) is stored, may be embedded and provided in the sleep monitoring and stimulation providing system 100 of the present disclosure or may be embedded in the mobile device 120. Alternatively, it may be an external device connected to the system 100 through a wired or wireless communication method.

Here, user state information may be mapped and stored for each of a plurality of users in the memory unit. For example, in the memory unit, physical information such as a height and weight of user A or identification information that can distinguish a plurality of users from one another may be mapped and stored for each user, and collected biometric information such as facial information, voice information, and heart rate information of user A may be mapped and stored.

Meanwhile, the calculation unit 150 is configured to control the entire system 100 in conjunction with the mobile device 120. For reference, the calculation unit 150 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software.

For reference, the calculation unit 150 may be excluded from the essential components depending on the embodiment. The system 100 may be designed such that the mobile device 120 already has a high-performance central processing unit that replaces the calculation unit 150.

On the other hand, although omitted in the diagram, the system 100 may additionally include a memory unit. According to an embodiment, the memory unit, like the calculation unit 150, may also be implemented to utilize a memory provided by the mobile device 120, but preferably, the system 100 may be designed to separately have a calculation unit and a memory unit.

The calculation unit 150 may analyze the user's sleep state based on the acquired user's biometric information.

The calculation unit 150 may monitor the user's current sleep state from at least one information item among the acquired user biometric information items, or a combination of a plurality of meaningful information items. For example, the user's sleep state or non-sleep state may be distinguished through the user's heart rate information (e.g., heart rate variability, etc.), respiration rate information (e.g., respiration variability, etc.), pulse rate information, movement information, and the like stored in the memory unit, and the sleep state or non-sleep state may be distinguished into more detailed stages.

Specifically, the calculation unit 150 may extract only valid data required for sleep state analysis by applying specific filtering conditions to the user's biometric information items stored in the memory unit, and may more accurately analyze the user's current sleep state based on the valid data. In this case, the user's biometric information may be extracted or the sleep state may be analyzed and monitored based on the user's sleep state information previously input by the user or a third party, or the user's sleep state information previously stored in the memory unit.

Furthermore, a result of the user's sleep state analyzed by the calculation unit 150 may be stored in the memory unit, and the user's sleep state may also be mapped and stored for each of a plurality of users. For example, sleep state information determined based on biometric information on user A or information on a predetermined threshold range that can determine a sleep/non-sleep state of user A may also be mapped and stored.

Here, according to the present disclosure, since biometric information, sleep state information, threshold range information, and the like are mapped and stored for each of a plurality of users together with the user's identification information in the memory unit, even if the sleep monitoring and stimulation providing system 100 according to the present disclosure is used in common by a plurality of users, biometric information collection, sleep state monitoring, and improvement may be accurately performed for each specific user.

In addition, according to the present disclosure, rather than simply storing the user's biometric information sporadically in the memory unit, sleep state information analyzed based on the user's biometric information may be mapped and stored together so as to allow the user's sleep state to be monitored more accurately based on the information stored in the memory unit, and user-specific sleep state information and a predetermined threshold range stored in the memory unit may be utilized so as to provide an effective improvement method for inducing a sleep state to the user.

That is, according to the present disclosure, the user's biometric information detected by the system 100 may be stored in the memory unit, and the user's sleep state information may be analyzed by the calculation unit 150 based on the user's biometric information stored in the memory unit, and the analyzed user's sleep state information may be stored again in the memory unit. Accordingly, the present disclosure may more accurately monitor the user's sleep state by utilizing user-specific biometric information and sleep state information stored in the memory unit, and may also generate a most effective stimulation signal to induce the user into a sleep state based on the monitored user sleep state information.

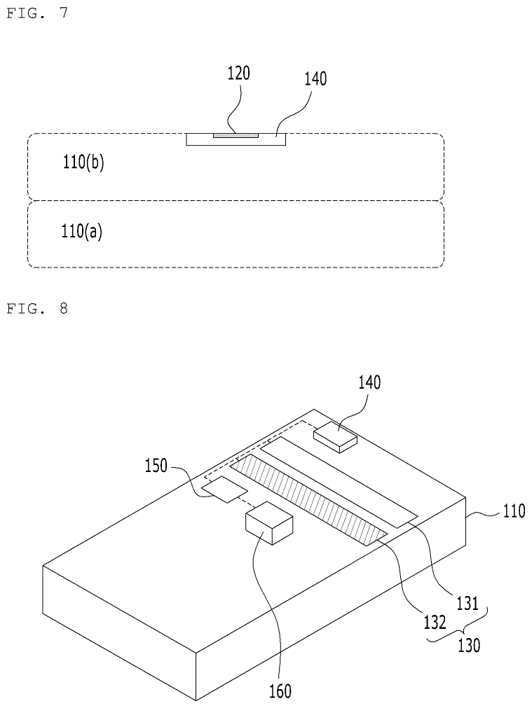

FIG. 6 is a diagram for explaining the system 100 according to a second embodiment of the present disclosure, and the second embodiment may be present in a state in which only the mounting unit 140 is provided in the layer unit 110, that is, the mattress. That is, the system 100 according to the second embodiment may be implemented so as to allow biosignal detection, sleep state analysis, and vibration generation control to be practically performed only by the mobile device 120, and it may be understood that the mobile device 120 constitutes the system 100 itself.

FIG. 7 shows a configuration in which the mounting unit 140 and the mobile device 120 are provided within the layer unit, and in this embodiment, since biosignal detection must be performed by the mobile device 120, the mounting unit 140 and the mobile device 120 may be provided in close contact with an upper surface of the uppermost layer 110(b) so as to be in contact with the user's body as much as possible. The mobile device 120 may be generally provided with an acceleration sensor, and the acceleration sensor, which is a sensor that detects a change in speed per unit time to recognize the movement of an object, and in this embodiment, the acceleration sensor may be utilized to detect a user's biosignal. Preferably, the position of the mounting unit 140 may be determined so as to allow the mobile device 120 to be disposed at a position close to the center of the user's heart (a position with the closest straight-line distance from the center of the heart), and more preferably, the position of the mounting unit 140 may be determined so as to allow an arbitrary surface of the mobile device 120 to be in close contact with the user's back. On the other hand, the mounting unit 140 may not necessarily be provided only on the layer, and the mobile device 120 may be disposed on the chest surface of the user lying down, more precisely, on the chest surface closest to the user's heart by manufacturing the mounting unit 140 in a form that the user can wear or put on. The acceleration sensor of the mobile device 120 disposed close to the user's heart in this manner may detect vibration generated by the heartbeat movement of the mobile device 120 in the form of accelerometer data, and the user's respiratory signal and the user's ballistic cardiogram signal may be extracted from the detected accelerometer data.

For reference, the position of the layer of the mounting unit 140 may be manufactured in a user-customized manner, and for example, at a stage of designing a mattress (layer), a position where the heartbeat movement signal is best detected when the user lies on the mattress may be determined in advance, and the mounting unit 140 may be disposed at that position, thereby effectively acquiring the user's biosignal.

Meanwhile, when extracting a respiratory signal and a cardiogram signal from the accelerometer data, a dedicated application (software) installed on the mobile device 120 may be used. Moreover, the dedicated application (software) may be provided with an artificial intelligence algorithm capable of learning, and thus a more accurate respiratory signal and cardiogram signal may be extracted through repeated analysis of accelerometer data.

In the foregoing description, only an example in which the acceleration sensor of the mobile device 120 detects a user's biosignal has been described, but when a detection means that can function as a vibration sensor or a pressure sensor in addition to the acceleration sensor is built into the mobile device 120, the user's biosignal may be detected when the user lies on the layer 110(b), and vibration stimulation may be provided to the user by a vibration means provided by the mobile device 120 itself.

In addition, in this embodiment, the mobile device 120 may analyze the user's sleep state based on the user's biosignal acquired by itself, and a vibration pattern may be varied according to a result of the analysis so as to transmit stimulation that can help improve sleep to the user.

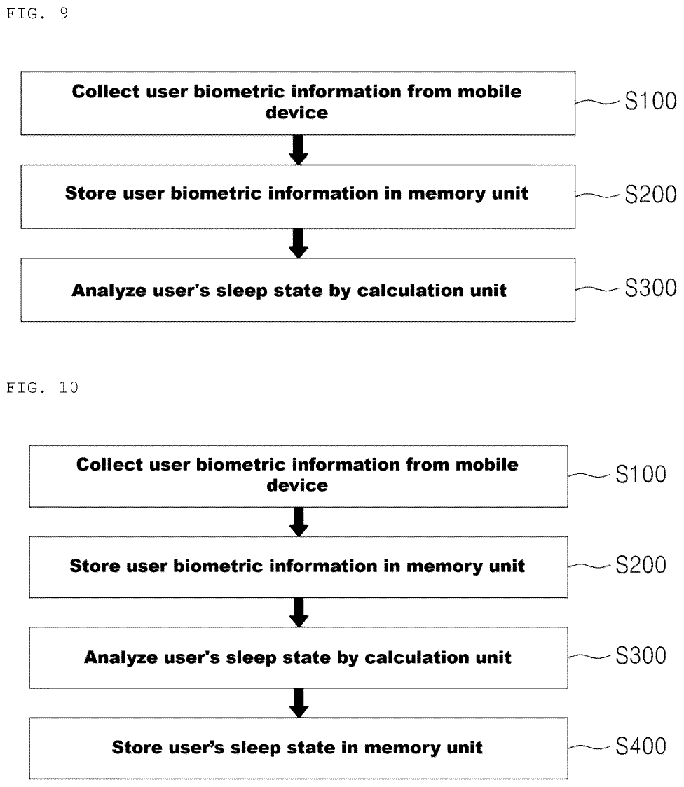

FIG. 8 is a diagram for explaining a sleep monitoring and stimulation providing system using a mobile device according to a third embodiment of the present disclosure, and hereinafter, a more detailed description will be provided with reference to FIG. 8.

Referring to FIG. 8, the system 100 according to a third embodiment of the present disclosure is similar to the system introduced in FIG. 1, but may further include a component of the stimulation generation unit 160 that generates a stimulation signal for inducing a user into a sleep state.

Here, the stimulation generation unit 160 may be configured to generate, when the user's sleep state analyzed by the calculation unit 150 or mobile device 120 exceeds or falls below a predetermined threshold range, a stimulation signal so as to allow the user's sleep state to fall within the predetermined threshold range.

The stimulation generation unit 160 generates, when the user's sleep state analyzed by the calculation unit 150 or mobile device 120 exceeds or falls below a predetermined threshold range corresponding to the user's sleep state information stored in the memory unit, a stimulation signal to induce the user into a sleep state. Here, a predetermined threshold range that can determine that the user is in a sleep state may be set based on the user's biometric information stored in the memory unit, and the stimulation generation unit 160 may generate a stimulation signal based on the user's biometric information stored in the memory unit and the user's sleep state information, but the stimulation signal may also be generated differently for each of a plurality of users.

The stimulation signal generated by the stimulation generation unit 160 may include at least one of a vibration signal and an acoustic signal, and the stimulation signal may provide a combination of stimulation signals to the user in conjunction with the mobile device 120. For example, vibration stimulation with a relatively greater intensity and shorter cycle may be generated by the stimulation generation unit 160, and vibration stimulation with a relatively lesser intensity and longer cycle may be generated by the mobile device 120 mounted on the mounting unit 140, thereby allowing various of stimulations to be transmitted to the user. Vibration stimulation from the stimulation generation unit 160 and acoustic stimulation from the mobile device 120 may be generated at the same time. For reference, it is shown in FIG. 8 that the stimulation generation unit 160 is provided in the center of the mattress, and the mounting unit 140 is provided at the head of a person based on the person's lying position, but it is understood that the position of the mounting unit 140 may vary.

Meanwhile, the vibration signal or the acoustic signal may be provided in a manner that can induce the user into a sleep state most quickly and effectively according to the user-specific sleep state information, and for example, only one of the vibration signal or the acoustic signal may be provided, or the vibration signal and the acoustic signal may be provided simultaneously, or the vibration signal or the acoustic signal may be provided alternately at a predetermined interval. That is, a stimulation signal generated by the stimulation generation unit 160 may be provided in various forms, such as a most suitable method for inducing the user into a sleep state or a preferred method set by the user, and the most suitable method for inducing the user into a sleep state may be set or recommended based on user-specific information stored in the memory unit.

In addition, the stimulation generation unit 160 may analyze the user's sleep state analyzed by the calculation unit 150 or mobile device 120 periodically or aperiodically to generate the stimulation signal by changing an intensity or a time period in real time. Furthermore, the stimulation generation unit 160 of the present disclosure periodically or aperiodically analyzes the user's sleep state analyzed by the calculation unit 150 or the mobile device 120, and stops generating the stimulation signal when the user's sleep state falls within a predetermined threshold range.

That is, according to the present disclosure, not only does the stimulation generation unit 160 generate and change a stimulation signal for inducing the user into a sleep state based on the user's sleep state analyzed by the calculation unit 150, but also does the calculation unit 150 analyze the user's sleep state periodically or aperiodically, when the user is determined to be within a predetermined threshold range based on a result of the stimulation signal, the stimulation generation unit 160 may stop generating the stimulation signal. Accordingly, the present disclosure may monitor the user's sleep state more accurately based on the collected user's biometric information, and generate a stimulation signal based on the monitored user's sleep state to induce the user into a sleep state, and stop, when the user is determined to be in a sleep state, generating the stimulation signal to maintain the sleep state without disturbing the user's sleep, thereby improving sleep activity.

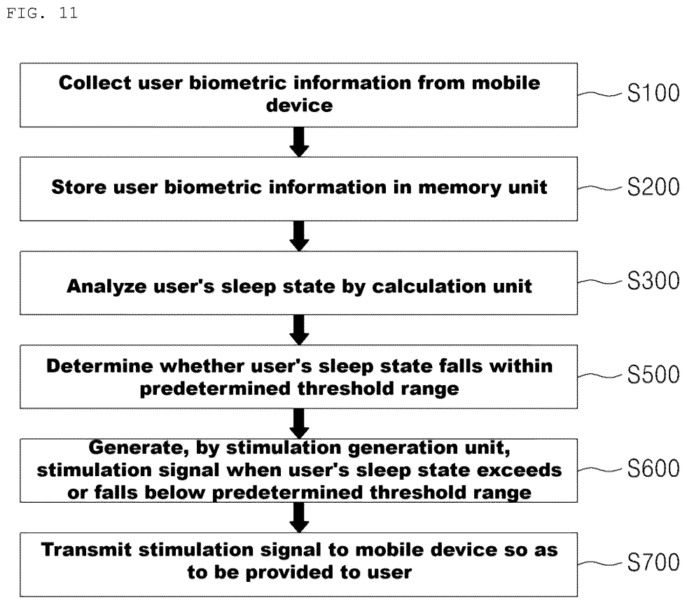

Hereinafter, a sleep monitoring and stimulation providing method using a mobile device according to an embodiment of the present disclosure will be described with reference to FIGS. 9 to 12.

FIG. 9 is a diagram for explaining a sleep monitoring and stimulation providing method using a mobile device according to an embodiment of the present disclosure.

Referring to FIG. 9, a sleep monitoring and stimulation providing method performed in a sleep monitoring and providing stimulation system using a mobile device including a layer unit, a mobile device, a memory unit, and a calculation unit includes collecting a user's biometric information from a detection unit provided in the layer unit (S100), storing the collected user's biometric information in the memory unit (S200), and analyzing the user's sleep state by the calculation unit based on the user's biometric information stored in the memory unit (S300).

Here, the collecting of the user's biometric information (S100) is performed while the user is lying and sleeping on the layer unit, and in particular, is performed through the detection unit when the mobile device is mounted on the mounting unit. The detection unit may include at least one sensor among a pressure sensor, a vibration sensor, a position sensor, an acoustic sensor, an infrared sensor, a motion sensor, a facial recognition sensor, a biometric recognition sensor, and a fingerprint recognition sensor. Specifically, the collecting of the user's biometric information (S100) may be performed in a manner of detecting the user's weight or location information through a pressure detection unit, or detecting the user's vibration signal through a vibration detection unit to acquire state information such as a heart rate, a breathing state, and a movement state, and may also be performed in a manner of recognizing the user's voice through an acoustic detection unit of the detection unit, or detecting the user's gesture through a motion detection unit of the detection unit to receive movement information.

Next, the storing of the user's biometric information in the memory unit (S200) is performed. The step S200 may be performed so as to allow the collected user biometric information to be mapped and stored for each of a plurality of users. For example, in the memory unit, physical information such as a height and weight of user A or identification information that can distinguish a plurality of users from one another may be mapped and stored for each user, and collected biometric information such as facial information, voice information, and heart rate information of user A may be mapped and stored. (See FIG. 5)

Then, based on the user's biometric information stored in the memory unit, the analyzing of the user's sleep state from the calculation unit (S300) is performed. The step S300 may be performed in a manner of monitoring the user's current sleep state from at least one of user biometric information items stored in the memory unit or from a combination of a plurality of meaningful information items in the step S200.

For example, the analyzing of the user's sleep state (S300) may distinguish a sleep state or non-sleep state by monitoring the user's sleep state through the user's heart rate information (e.g., heart rate variability, etc.), respiration rate information (e.g., respiration variability, etc.), pulse rate information, movement information, and the like stored in the memory unit, and the sleep state or non-sleep state may be distinguished into more detailed stages.

Specifically, the analyzing of the user's sleep state (S300) may further include a process in which the calculation unit applies specific filtering conditions to the user's biometric information items stored in the memory unit to extract only valid data required for sleep state analysis, and the user's current sleep state may be analyzed more accurately based on the extracted valid data. In this case, the user's biometric information may be extracted or the sleep state may be analyzed and monitored based on the user's sleep state information previously input by the user or a third party, or the user's sleep state information previously stored in the memory unit.

That is, as another embodiment of the present disclosure, subsequent to the analyzing of the user's sleep state (S300), the analyzed user's sleep state may be stored in the memory unit so as to be utilized for analyzing the user's sleep state.

FIG. 10 is a diagram for explaining a monitoring sleep and stimulation providing method using a mobile device according to another embodiment of the present disclosure, and as shown in FIG. 10, subsequent to analyzing the user's sleep state by the calculation unit (S300), storing the user's sleep state analyzed by the calculation unit in the memory unit (S400) may be performed.

Here, the step S400 may also be performed so as to allow the user's sleep state to be mapped and stored for each of a plurality of users, and furthermore, sleep state information determined based on biometric information of user A or information on a predetermined threshold range that can determine a sleep/non-sleep state of user A may also be mapped and stored. (See FIG. 5)

That is, according to the present disclosure, since biometric information, sleep state information, threshold range information, and the like are mapped and stored for each of a plurality of users together with the user's identification information in the memory unit, even if a plurality of users use a common mattress simultaneously or sequentially, biometric information collection, sleep state monitoring, and improvement may be accurately performed for each specific user.

In addition, according to the present disclosure, rather than simply storing the user's biometric information sporadically in the memory unit, sleep state information analyzed based on the user's biometric information may be mapped and stored together so as to allow the user's sleep state to be monitored more accurately based on the information stored in the memory unit, and user-specific sleep state information and a predetermined threshold range stored in the memory unit may be utilized so as to provide an effective improvement method for inducing a sleep state to the user.

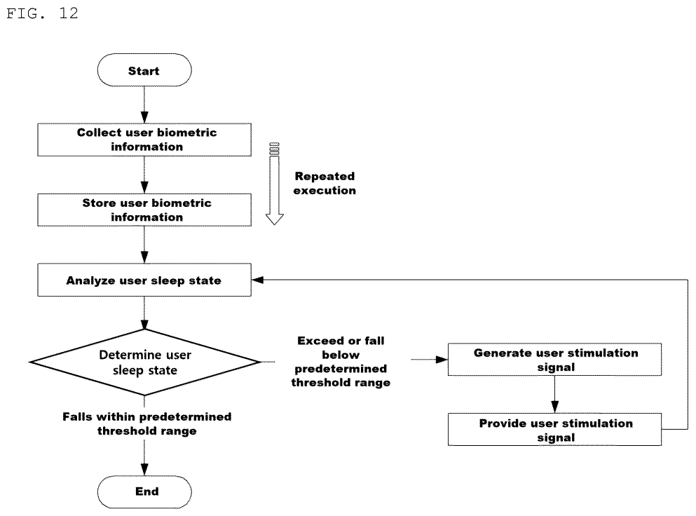

FIG. 11 is a diagram for explaining a sleep monitoring and stimulation providing method using a mobile device according to still another embodiment of the present disclosure, and hereinafter, with reference to FIG. 11, a method of generating a stimulation signal for inducing a user into a sleep state by utilizing the user's biometric information and sleep state information stored in a memory unit will be described.

Referring to FIG. 11, subsequent to the analyzing, by the calculation unit, of the user's sleep state (S300), the method may further include determining, by the calculation unit, whether the user's sleep state falls within a predetermined threshold range (S500), generating, by a stimulation generation unit, a stimulation signal so as to allow the user's sleep state to fall within the predetermined threshold range when the user's sleep state exceeds or falls below the predetermined threshold range (S600), and transmitting the stimulation signal generated by the stimulation generation unit to a mobile device so as to be provided to the user (S700).

The determining of whether the user's sleep state falls within a predetermined threshold range (S500) is performed based on the user's predetermined threshold information stored in the memory unit. The predetermined threshold information, which is set based on the user's biometric information stored in the memory unit, may refer to a range of biometric information that can determine that the user is in a sleep state, and may also be set for each of a plurality of users.

Next, when it is determined in the step S500 that the user's sleep state exceeds or falls below the predetermined threshold range, generating, by the stimulation generation unit, a stimulation signal (S600) is performed. The step S600 may be generated based on the user's biometric information stored in the memory unit, but may be generated so as to allow the user's biometric information to fall within a preset or the user's predetermined threshold range stored in the memory unit (a range of biometric information that can determine that the user is in a sleep state), and may be generated for each of a plurality of users.

Here, the stimulation signal generated in the step S600 may include at least one of a vibration signal and an acoustic signal, and the vibration signal or the acoustic signal may be provided to the user by the components/functions provided in the mobile device.

The vibration signal or the acoustic signal may be provided in a manner that can induce the user into a sleep state most quickly and effectively according to the user-specific sleep state information, and for example, only one of the vibration signal or the acoustic signal may be provided, or the vibration signal and the acoustic signal may be provided simultaneously, or the vibration signal or the acoustic signal may be provided alternately at a predetermined interval. That is, a stimulation signal generated by the stimulation generation unit may be provided in various forms, such as a most suitable method for inducing the user into a sleep state or a preferred method set by the user, and the most suitable method for inducing the user into a sleep state may be set or recommended based on user-specific information stored in the memory unit.

In addition, subsequent to a process of generating and providing the user stimulation signal, a process of collecting and storing the user's biometric information may be repeatedly performed periodically or aperiodically through a mobile device, and as a result of the repeated execution, the user's sleep state analysis result may be updated, and according to the updated user's sleep state analysis result, an intensity or a time period of the user stimulation signal generated from the stimulation generation unit may be changed in real time. Specifically, when it is determined that the user's biometric information collected and analyzed as a result of providing the user stimulation signal is getting closer to a sleep state, the method may be changed to reduce the intensity and time period of the stimulation signal being provided, and when it is determined that the user's biometric information collected and analyzed as a result of providing the user stimulation signal is getting farther from a sleep state or is not significantly different from before, the method may be changed to strengthen the intensity and time period of the stimulation signal being provided.

That is, according to the present disclosure, in addition to monitoring the user's sleep state based on the user's biometric information collected through a mobile device, the present disclosure may further include generating a stimulation signal for inducing the user into a sleep state, and in particular, the generating of the stimulation signal may be performed based on a predetermined threshold range set based on the user's biometric information collected through the mobile device, that is, a biometric information range that can determine that the user is in a sleep state.

Accordingly, the present disclosure may not only collect biometric information of a user who is lying and sleeping on a mattress through a mobile device, but also monitor the user's sleep state more accurately based on the collected biometric information and generate an optimal stimulation signal that can induce the user into a sleep state so as to improve the user's sleep state, wherein a process of collecting biometric information and providing a stimulation signal is performed using a mobile device that does not disturb the user's sleep.

FIG. 12 is a diagram for more specifically explaining a sleep monitoring and stimulation provision method using a mobile device according to still another embodiment of the present disclosure.

Referring to FIG. 12, a process of collecting biometric information of a user lying on the mattress through a mobile device and storing the collected user biometric information in a memory unit is repeatedly performed. The process may be performed periodically or aperiodically, and may be utilized to more accurately collect user biometric information to monitor the user's sleep state or generate a stimulation signal to induce the user's sleep state.

Next, a monitoring process is performed in which the user's sleep state is analyzed based on the user's biometric information stored in the memory unit. The process may be performed by analyzing the user's heart rate information, respiration rate information, movement information, and the like, and may be accumulated, updated and analyzed through the previously repeated process of collecting and storing the user's biometric information.

Then, a process of determining the analyzed user's sleep state is performed. The process of determining the user's sleep state may be performed so as to be determined based on whether the user's biometric information falls within a preset threshold range. The threshold range refers to a range of biometric information that can determine that the user is in a sleep state, and may be mapped and set for each of a plurality of users.

As a result of determining the user's sleep state, when the user's biometric information falls within a predetermined threshold range, the user may be determined to be currently in a sleep state, and no separate stimulation will be provided to the user. However, the process of collecting and storing the user's biometric information may be repeatedly performed, periodically or aperiodically, through a mobile device, and a stimulation signal may be generated when the user exceeds or falls below a predetermined threshold range as a result of the repeated execution.

As a result of determining the user's sleep state, when the user's biometric information exceeds or falls below a predetermined threshold range, the user may be determined to be currently in a non-sleep state, and a stimulation signal that can induce the user into a sleep state is generated. The stimulation signal may include at least one of a vibration signal and an acoustic signal, and the vibration signal or the acoustic signal may be transmitted to the mobile device and provided to the user in the form of at least one of the vibration signal or the acoustic signal by utilizing the components/functions provided in the mobile device.

Specifically, the vibration signal or acoustic signal may be provided in a manner that can most quickly and effectively induce the user into a sleep state according to user-specific sleep state information, and may be generated differently for each of a plurality of users.

For example, only one of a vibration signal or an acoustic signal may be provided, or both a vibration signal and an acoustic signal may be provided simultaneously, or a vibration signal or an acoustic signal may be provided alternately at a predetermined interval. That is, a stimulation signal generated by the stimulation generation unit may be provided in various forms, such as a most suitable method for inducing the user into a sleep state or a preferred method set by the user, and the most suitable method for inducing the user into a sleep state may be set or recommended based on user-specific information stored in the memory unit.

In addition, subsequent to a process of generating and providing the user stimulation signal, a process of collecting and storing the user's biometric information may be repeatedly performed periodically or aperiodically through a mobile device, and as a result of the repeated execution, an intensity or a time period of the user stimulation signal generated from the stimulation generation unit may be changed in real time depending on the analyzed user's sleep state.

That is, when it is determined that the user's biometric information collected and analyzed as a result of providing the user stimulation signal is getting closer to a sleep state, the method may be changed in a manner of reducing the intensity and time period of the stimulation signal being provided, and when it is determined that the user's biometric information collected and analyzed as a result of providing the user stimulation signal is getting farther from a sleep state or is not significantly different from before, the method may be changed to strengthen the intensity and time period of the stimulation signal being provided.

In other words, the present disclosure may collect user biometric information through a mobile device and provide a stimulation signal for inducing the user into a sleep state, and repeatedly perform this process periodically/aperiodically, thereby having an advantage capable of monitoring the user's sleep state without disturbing the user's sleep, and generating and changing a stimulation signal for inducing the user into a sleep state in real time.

In the above, a sleep monitoring stimulation providing system using a mobile device, and a method thereof have been described. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

1. A sleep analysis segment detection system using a mobile device, the system comprising:
at least one layer having a plurality of components;
a detection unit for detecting a biosignal of a user lying on the layer unit;
a mounting unit located inside or outside the layer unit to mount a mobile device;
a memory unit in which the user's biosignal collected by the detection unit is stored; and
a calculation unit that analyzes the user's sleep state based on the user's biometric information stored in the memory unit,
wherein the calculation unit, the detection unit, and the memory unit are operated while the mobile device mounted on the mounting unit is in conjunction with the calculation unit.

2. The system of claim 1, wherein the mobile device is provided in a form that can be mounted and detached on a surface or inside of the mounting unit.

3. A sleep monitoring and stimulation providing system using a mobile device, the system comprising:
at least one layer unit; and
a mounting unit located inside the layer unit and configured to mount a mobile device,
wherein a biosignal of a user lying on the layer unit is detected by the mobile device mounted on the mounting unit, or vibration stimulation is generated in a direction of an upper surface of the layer unit.

4. The system of claim 3, wherein the mounting unit is provided at a position in contact with an uppermost surface of the layer unit.

5. A sleep monitoring and stimulation providing method performed by a sleep monitoring and stimulation providing system using a mobile device comprising a layer unit, a detection unit for detecting a user's biosignal, a mobile device mounted on a mounting unit, a memory unit, and a calculation unit, the method comprising:
collecting biometric information of a user lying on the layer unit by the detection unit;
storing the user's biometric information in the memory unit;
analyzing the user's sleep state in the calculation unit based on the user's biometric information stored in the memory unit; and
vibrating the mobile device to generate vibration stimulation according to the user's sleep state.

6. A sleep monitoring and stimulation providing method performed by a sleep monitoring and stimulation providing system using a mobile device comprising a layer unit and a mobile device mounted on a mounting unit, the method comprising:
collecting biometric information of a user lying on the layer unit by the mobile device;
storing the user's biometric information in the mobile device;
analyzing the user's sleep state based on the user's biometric information by the mobile device; and
vibrating the mobile device according to the user's sleep state to generate vibration stimulation.
